(19) United States
(12) Patent Application Publication
(10) Pub. No.: US 2016/0100985 A1
(43) Pub. Date: Apr. 14, 2016

(54) REUSABLE EYE PATCH

(71) Applicants: Julia M. Kennedy, Racine, WI (US); Gary Lee Kennedy, Racine, WI (US)

(72) Inventors: Julia M. Kennedy, Racine, WI (US); Gary Lee Kennedy, Racine, WI (US)

(21) Appl. No.: 14/512,134

(22) Filed: Oct. 10, 2014

(51) Int. Cl. A61F 9/04

(52) U.S. Cl. CPC A61F 9/04

(57) ABSTRACT

An eye patch with a slim profile having a first side and a second side, the first side including a circular concave portion and the second side having a corresponding circular convex portion, the concave and convex portions each fully surrounded by an edge portion, both sides being flexible and made of a non-allergenic, elastomeric material thereby facilitating protection of a user's eye from debris or other foreign material while also allowing the eye patch to be reusable and washable.

### FIELD

This application relates to medical devices and, more particularly, to reusable eye patches.

### BACKGROUND

Eye patches have been utilized for a variety reasons. In most cases eye patches are worn for medical reasons such as amblyopia, diplopia, Bell's palsy and other eye ailments or to protect eyes that are injured, missing or disfigured. Most of these eye ailments require the use of a traditional eye patch that blocks light from the eye which is covered by the eye patch. Typically, these types of eye patches use an adhesive to keep the eye patch in place on the user's face while it is worn. Eye patches of the prior art are generally made of fabric and an adhesive is used around the outer edges of the eye patch. Other types are eye patches are disposable and are simply worn once and then discarded. Some eye patches of the prior art use an elastic band or other type of band to secure the eye patch in place.

Examples of eye patches that are made of various fabric materials, block light from the user's eye, and use an adhesive to secure the eye patch in place are disclosed in the following United States patents: U.S. Pat. No. 4,944,040 (Riedel, et al.), U.S. Pat. No. 4,995,114 (Price, Jr.) and U.S. Pat. No. 8,683,613 (Van Dalen, et al.) as well as EP 2,736,466 (Awan).

There are a number of shortcomings with eye patches of the prior art. One such shortcoming is that eye patches of the prior art are generally made of fabric. Fabric can easily get dirty from make-up, body oils and the environment. Eye patches of this type also generally have seams or edges due to their construction which can rub and chafe and result in user discomfort. Eye patches made of any sort of fabric can also be difficult to clean and are also unable to be effectively and routinely sanitized. Routine cleaning of fabric eye patches can also result in breakdown of the fabric. There is a need for a slim-profile, flexible eye patch which is not made of a fabric material so it is able to be worn multiple times as well as able to be routinely sanitized without effecting the strength and durability of the eye patch material.

Eye patches of the prior art are generally focused on preventing as much light as possible from reaching the eye which is covered by the eye patch. However, there are some conditions in which it would be beneficial to have a physical barrier over the eye (in the form of an eye patch) but also allow the user to see out of the covered eye by way of a transparent eye patch. One such situation would be a person recovering from an eye injury that needs to protect the eye from foreign debris until it heals but also needs to see out of the effected eye. Another such situation occurs in individuals which have eyelids that do not close properly (one such example is Bell's palsy). With these conditions, salves and ointments are administered to the eye to keep it moist and protected. An eye patch that would keep debris from sticking to the salves and ointments would be beneficial. It would also be advantageous to have an eye patch that is able to be easily cleaned so that medication residue can be removed from the eye patch. Therefore, it would be desirable to have an eye patch which protects the eye and eye medications from foreign material and also allows the user to see out of the eye covered by the eye patch.

Some eye patches of the prior art are also not shaped to allow the eye or eyelashes covered by the eye patch to move freely. Other eye patches of the prior art are not slim enough to fit under a user's glasses. It would be desirable to have an eye patch with a slim profile that can fit under glasses, is shaped to allow the eye and eyelashes free range of movement and also effectively covers the eye and eyelashes.

A wide variety of eye patches have been created and are available. However, there is a need for improvement in eye patches, and it is to this need that this device is directed.

This device overcomes certain problems and shortcomings in the prior art, including those mentioned above and others by namely providing an improved eye patch.

### SUMMARY

In accordance with the present device, an eye patch is provided for treatment of various issues associated with the eye. The eye patch of this invention overcomes certain problems and shortcomings of the prior art and provides a unique structure satisfying eye treatment needs. The eye patch is reusable, washable and has a slim profile. The eye patch includes a first side and a second side, the first side including a circular concave portion and the second side having a corresponding circular convex portion, the concave and convex portions each fully-surrounded on all sides by an edge portion, both sides being flexible and made of a transparent, non-allergenic, elastomeric material thereby facilitating protection of a user's eye from debris or other foreign material while also allowing the user to see with the eye covered by the eye patch.

In highly preferred embodiments, the concave portion is shaped to allow the user's eye and eyelashes unrestricted movement when the user is wearing the eye patch. It is highly preferred that the non-allergenic, elastomeric material is a medical- grade silicone. It is also highly preferred that the thickness of the eye patch is between 0.30-0.40 inches.

In preferred embodiments, the eye patch is waterproof as well as weather resistant. In other preferred embodiments, an elastic strap is used for securement of the eye patch over the user's eye.

In some highly preferred embodiments the eye patch is made of an opaque, non-allergenic, elastomeric material such as medical-grade silicone thereby facilitating protection of a user's eye from debris or other foreign material while also allowing the eye patch to be reusable and washable.

### BRIEF DESCRIPTION OF THE DRAWINGS

FIG. 1 is a perspective view of a reusable, slim-profile eye patch made of an opaque material and secured over a user's eye.

FIG. 2 is a front view of the reusable, slim-profile eye patch of FIG. 1.

FIG. 3 is a side view of the reusable, slim-profile eye patch of FIG. 1.

FIG. 4 is a back view of the reusable, slim-profile eye patch of FIG. 1.

FIG. 5 is a cross-section of the reusable, slim-profile eye patch taken along line 5-5 of FIG. 2.

FIG. 6 is perspective view of the reusable, slim-profile eye patch made of transparent material and secured over a user's eye.

FIG. 7 is front view of a reusable, slim-profile eye patch of FIG. 6 made of an transparent material.

### DETAILED DESCRIPTION OF PREFERRED EMBODIMENTS

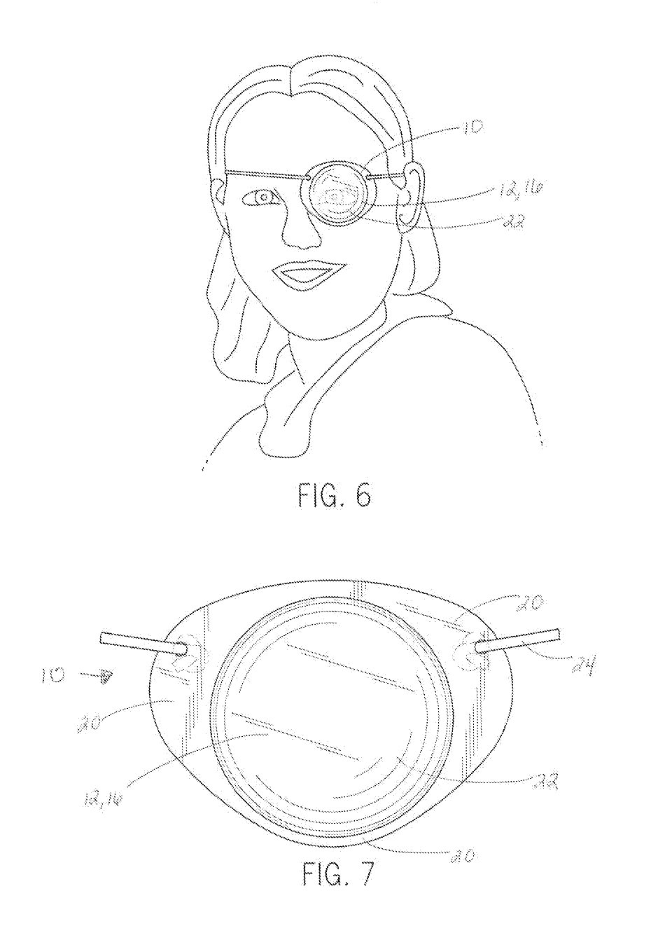

FIGS. 1-7 illustrate an inventive method for a reusable and washable eye patch 10. As seen in FIGS. 1 and 6, eye patch 10 can be made of an opaque 26 or transparent 22 material. Use of an opaque material 26 is useful to treat certain eye conditions such as amblyopia when it is imperative that light not reach the user's eye while eye patch 10 is in place as shown in FIGS. 1-5. Treatment of other eye conditions require the user's eye be safeguarded from debris but do not require that light to be blocked from the eye when eye patch 10 is in use. Use of a transparent material 22 for eye patch 10 is seen best in FIGS. 6-7 for such uses. Eye patch 10 made of transparent material 22 allows user to see out of the eye when eye patch 10 is secured over the respective eye.

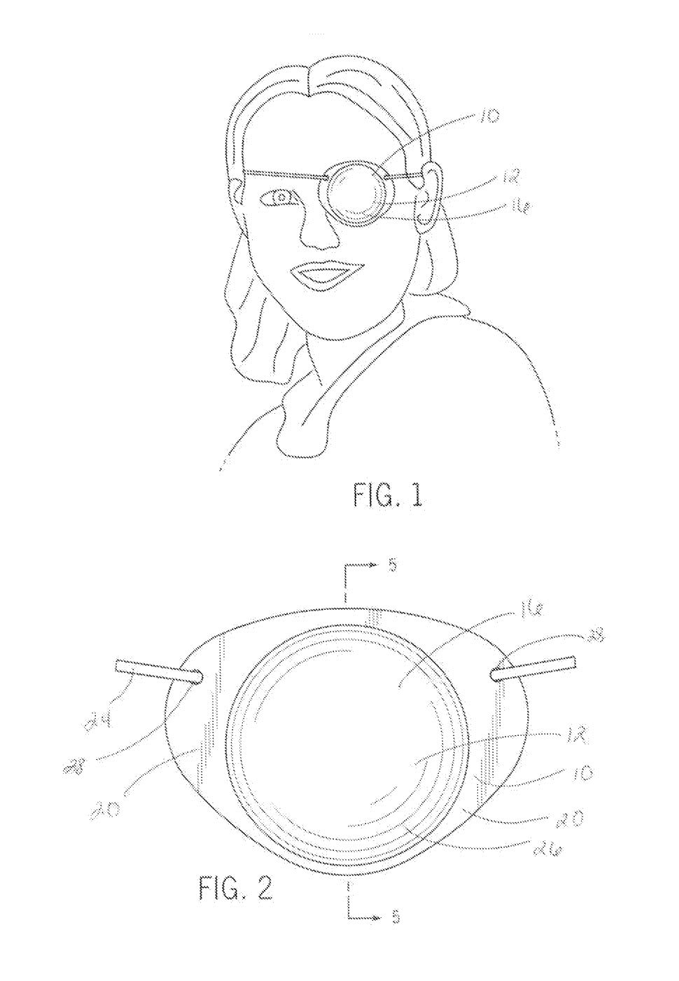

FIG. 2 illustrates that eye patch 10 has a first side 12 and FIG. 4 illustrates that eye patch 10 also includes a corresponding second side 14. First side 12 has a circular concave portion 16 and second side 14 has a corresponding circular convex portion 18. As shown in FIGS. 1-7, concave 16 and convex 18 portions are each fully-surrounded by edge portion 20. The amount of edge portion 20 around concave portion 16 and convex portion 18 can vary but at least some portion of edge portion 20 always surrounds both concave portion 16 and convex portion 18.

Both first side 12 and second side 14 are flexible. In some embodiments, as seen best in FIGS. 6-7, eye patch 10 is made of a transparent, non-allergenic, elastomeric material thereby facilitating protection of a user's eye from debris or other foreign material while also allowing the user to see with the eye covered by eye patch. 10. Other embodiments of eye patch 10, include eye patch 10 being made of an opaque material as seen in FIGS. 1-5.

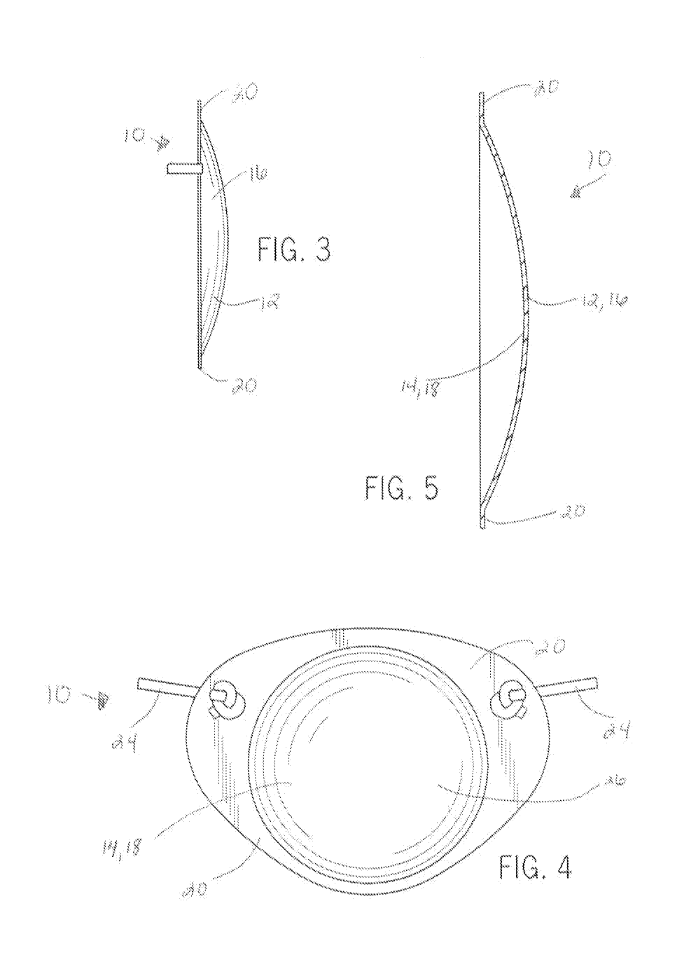

FIGS. 3 and 5 illustrate best that eye patch 10 has a slim profile. The thickness of eye patch 10 as seen in the cross section of eye patch 10 in FIG. 5 is between 0.30-0.40 inches. Concave portion 16 and corresponding convex portion 18 are shaped to allow user's eye and eyelashes unrestricted movement when user is wearing eye patch 10. The slim profile of eye patch 10 also allows eye patch 10 to fit under glasses in many cases. This is advantageous as it allows vision to be corrected in a user's remaining useable eye.

Eye patch 10 is made of a non-allergenic, elastomeric material such as a medical-grade silicone, however, any non-allergenic, elastomeric malleable product can be used. Use of a medical-grade silicone provides the benefit of either blocking light from the eye or allowing the user to see out of eye covered by eye patch 10 when in use (depending on the medical needs of the user) and also provides an eye patch 10 that will not irritate the skin of the user due to an allergy. Silicone is also an ideal material for eye patch 10 because it does not have seams which can rub and irritate a user's skin or even eye while in use.

Eye patch 10 made of silicone can also be easily cleaned by the user by hand or in the dishwasher as eye patch 10 is durable enough for high-heat sanitizing. Oftentimes salves and ointments are administered to the eye to keep it moist and protected. Ointments and other medications can be easily cleaned off of eye patch 10 by the methods described above or by simply using alcohol swabs and/or prepackaged alcohol moistened pads.

Eye patch 10 can be manufactured by either injection molding or vacuum forming. Eye patch 10 is flexible yet rigid enough that it will hold its shape over years of constant daily use and frequent cleaning. The size of eye patch 10 can vary by the size of user's eye and head. Different types of eye patch 10 are available to accommodate both children and adults. For treatment of conditions that require light to be blocked from the eye, the size of eye patch 10 is made to be sufficient to cover the eye and block out most light.

Eye patch 10 is weather resistant and can withstand extreme heating and cooling temperature changes due to weather including well-below freezing temperatures over an extended period of time. Eye patch 10 is also waterproof.

Elastic strap 24 as seen in FIGS. 1-7 is for securement of eye patch 10 over user's eye. Strap 24 can be made of varying widths of elastic and its length is adjustable. Holes 28 are designed to hold strap 24 in place and to assist in keeping eye patch 10 over user's eye. Strap 24 can be easily replaced when it wears out by simply threading a new strap 28 (which can be purchased at any sewing or craft store) through holes 28. When strap 24 wears out eye patch 10 does not need to be discarded, strap 24 simply needs to be replaced, this is both economical and ecologically responsible.

Reusable eye patch 10 provides a durable, reusable, low-cost and low- maintenance eye patch.

While the principles of this invention have been described in connection with specific embodiments, it should be understood clearly that these descriptions are made only by way of example and are not intended to limit the scope of the invention.

1. A reusable and washable eye patch with a slim profile comprising a first side and a second side, the first side including a circular concave portion and the second side having a corresponding circular convex portion, the concave and convex portions each fully surrounded by an edge portion, both sides being flexible and made of a transparent, non-allergenic, elastomeric material thereby facilitating protection of a user's eye from debris or other foreign material while also allowing the user to see with the eye covered by the eye patch.

2. The eye patch of claim 1 wherein the concave portion is shaped to allow the user's eye and eyelashes unrestricted movement when the user is wearing the eye patch.

3. The eye patch of claim 1 wherein the non-allergenic, elastomeric material is a medical-grade silicone.

4. The eye patch of claim 1 wherein the thickness of the eye patch is between 0. 30-0.40 inches.

5. The eye patch of claim 1 wherein the eye patch is weather resistant.

6. The eye patch of claim 1 wherein the eye patch is waterproof.

7. The eye patch of claim 1 further including an elastic strap for securement of the eye patch over the user's eye.

8. An eye patch with a slim profile comprising a first side and a second side, the first side including a circular concave portion and the second side having a corresponding circular convex portion, the concave and convex portions each fully surrounded by an edge portion, both sides being flexible and made of an opaque, non- allergenic, elastomeric material thereby facilitating protection of a user's eye from debris or other foreign material while also allowing the eye patch to be reusable and washable.

9. The eye patch of claim 8 wherein the concave portion is shaped to allow the user's eye and eyelashes unrestricted movement when the user is wearing the eye patch.

10. The eye patch of claim 8 wherein the non-allergenic, elastomeric material is a medical-grade silicone.

11. The eye patch of claim 8 wherein the thickness of the eye patch is between 0. 30-0.40inches.

12. The eye patch of claim 8 wherein the eye patch is weather resistant.

13. The eye patch of claim 8 wherein the eye patch is waterproof.

14. The eye patch of claim 8 further including an elastic strap for securement of the eye patch over the user's eye.

15. The eye patch of claim 8 wherein the eye patch is universal and can be used on either the user's left eye or right eye.
